(19) United States
(12) Patent Application Publication
(10) Pub. No.: US 2008/0299172 A1
(43) Pub. Date: Dec. 4, 2008

(54) TISSUE REPAIR IMPLANT

(76) Inventors: Stuart Young, Del Mar, CA (US); Neville Alleyne, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/132,557

(22) Filed: Jun. 3, 2008

Related U.S. Application Data
(60) Provisional application No. 60/941,906, filed on Jun. 4, 2007.

(51) Int. Cl. A61K 9/70; A61P 17/00

(52) U.S. Cl. 424/423

(57) ABSTRACT

An implant is formed from a sheet material and a plurality of microparticles. The sheet may be coated with a mixture of collagen and PMMA beads. The implant may be used to treat many types of defects, including hernias, skin defects, tendon defects, and ulcers. A biocompatible alloplastic mesh implant is stronger and more resistant to infection than typical mesh implants.

### CROSS REFERENCE TO RELATED APPLICATIONS

This application claims priority under 35 U.S.C. Section 119(e) to Provisional Application 60/941,906, filed on Jun. 4, 2007, the disclosure of which is hereby incorporated by reference in its entirety.

### BACKGROUND OF THE INVENTION

1. Field of the Invention

The present invention relates generally to surgical implants, and more particularly to tissue repair implants.

2. Description of the Related Art

Tissue repair implants are surgical implants that often have a mesh configuration. These implants may be used to strengthen a surgical repair, such as a repair of abdominal or ventral hernias where there is a weakness in the abdominal wall or in the floor of the inguinal canal. In addition, such implants are used to treat venostasis ulcers, diabetic ulcers, or skin defects that may have occurred due to trauma, buns, or wounds that cannot be approximated. Mesh implants are also used to wrap a graft for containment after procedures such as ACL reconstruction or tendon repair surgery.

A variety of materials have been utilized for these implants, including metal, various polymers such as nylon, polypropylene, as well as many others. Collagen mesh has also been utilized. U.S. Pat. No. 7,060,103, for example, describes a resorbable collagen scaffold derived from the intestinal submucosa.

No particular mesh graft material has been universally accepted, and continued improvements in these materials would be beneficial.

### SUMMARY OF THE INVENTION

The system, methods, and devices of the invention each have several aspects, no single one of which is solely responsible for its desirable attributes. Without limiting the scope of this invention, its more prominent features will now be discussed briefly. After considering this discussion, and particularly after reading the section entitled “Detailed Description of the Preferred Embodiments” one will understand how the features of this invention provide advantages over other mesh implants.

In one embodiment, an implant comprises a sheet material and a plurality of microparticles in association with the sheet material. The implant may include collagen and the microparticles may comprise PMMA beads.

In other embodiments, methods of treating an ulcer, a skin defect, and/or a hernia comprise applying a microparticle containing sheet material to the ulcer, skin defect, and/or hernia.

In another embodiment, a method of making an implant comprises coating a sheet material with a substance comprising a plurality of microparticles in a suspending agent.

Other aspects and advantages of the present invention will become apparent from the following detailed description, which, when taken in conjunction with the accompanying drawings, illustrates by way of example the principles of the invention.

### DETAILED DESCRIPTION OF THE PREFERRED EMBODIMENTS

The following detailed description is directed to certain specific embodiments. However, the invention can be embodied in a multitude of different ways. Reference in this specification to “one embodiment” or “an embodiment” means that a particular feature, structure, or characteristic described in connection with the embodiment is included in at least one embodiment. The appearances of the phrase “in one embodiment,” “according to one embodiment,” or “in some embodiments” in various places in the specification are not necessarily all referring to the same embodiment, nor are separate or alternative embodiments mutually exclusive of other embodiments. Moreover, various features are described which may be exhibited by some embodiments and not by others. Similarly, one or more features may be described for one embodiment which can also be reasonably used in another embodiment.

Preferred embodiments comprise a sheet that can be made from a wide variety of materials. These can include permanent and bioabsorbable materials such as metals and various polymers that are already in use as tissue support structures. Collagen fiber may also be used. The fibrous sheet is formed or impregnated with a biocompatible alloplastic material that preferably comprises a plurality of particles. These particles can comprise solid microparticles in representative embodiments. In some implementations, the microparticles may not be altogether solid, such as implementations involving hollow or porous microparticles. As used herein, the term “microparticles” refers to particles (e.g., in a dust or powder form) possessing an average diameter of 500 microns or less. Typically, the average diameter will be greater than about 20 microns, rendering the microparticles too large to be “eaten” by monocytes. The microparticles can have diameters sufficient to keep them from being washed away through lymph tracts or other tissue tracts from the implantation site. If the microparticles do not have a spherical form, then the diameter as used herein refers to the greatest diameter of the smallest cross sectional area. It is, however, also possible to use smaller microparticles ranging from 4 to 5 microns or 5 to 10 microns in diameter. Typically, the microparticles will have an average diameter less than about 200 microns. In representative embodiments, the microparticles can have an average diameter of about 15 to about 200 microns and in certain implementations from about 15 to about 60 microns. In some embodiments, the particles comprise polymethylmethacrylate (“PMMA”) particles. The particles may be suspended in a collagen matrix. The impregnation of the mesh with a material comprising this essentially permanent particulate material can provide additional support to the tissues and enhance tissue ingrowth and natural collagen formation in and around the mesh material.

The composition the mesh is impregnated with may also include lidocaine. In one embodiment, the collagen is bovine collagen. In one embodiment, the suspending agent has 0.3% lidocaine, such that PMMA microspheres are suspended in a bulking agent of collagen and lidocaine which is made part of the mesh implant.

The ratio of the particles to the suspending agent (e.g. collagen) may be 1 to 2. In one embodiment, the ratio of the particles to the suspending agent may be in the range of 1 to 1 to 1 to 5. In principle, it is possible to use any inert histocompatible polymer for producing the microparticles. Embodiments may comprise, in whole or in part, non-polymer microparticles. In an exemplary embodiment, the implant comprises one or more of the materials described under the name Artefill® and obtainable at www.artefill.com. Exemplary embodiments are also described in the U.S. Pat. No. 5,344,452, the entire contents of which is incorporated herein by reference.

The impregnation material may comprise, for example, about 20% substantially smooth spherical PMMA beads ranging in size from about 32-40 micrometer diameter, and with low levels of methylmethacrylate monomer impurities. The remaining 80% may comprise a solution of partially denatured collagen, which may be about 3.5% collagen in a solution of water and/or alcohol. In one embodiment, there are about 6 million particles per cc of implant material. The sheet may be soaked in or coated with such a collagen/PMMA bead solution to produce these embodiments of the invention.

It can be advantageous for the microparticles used to have a smooth surface and be free from corners and edges, such that the microparticles don't have sharp transitions on their surfaces. In addition they may not have peaks of any kind or tapered projections. According to one implementation, the surface does not have pores. In another implementation, the surfaces may comprise pores. Although smooth, and especially spherical particles can be advantageous, in some embodiments, non-smooth microparticles with corners or peaks or the like may be used.

The implants may be made out of sheets of varying sizes, thicknesses, and geometries. In one embodiment, the biocompatible alloplastic mesh implant has a rectangular shape. For a hernia repair, the dimensions of a rectangular biocompatible alloplastic mesh implant are likely to be between 50 mm×100 mm×1 mm and 100 mm×200 mm×5 mm, with the optimal dimensions being 4 mm×80 mm×3 mm. In another embodiment, the biocompatible alloplastic mesh implant has a circular shape. For treatment of umbilical hernias, a circular implant is likely to have a radius between 20 mm and 80 mm and a thickness between 1 mm and 10 mm. Optimally, the umbilical hernia implant will have a radius of 20 mm and a thickness of 3 mm.

In one embodiment, the biocompatible alloplastic mesh implant is a solid sheet. Woven structures are advantageous, as well as microporous materials. The implant may be fenestrated to allow additional fibrovascular infiltration through the mesh scaffold. The fenestrations may be formed in a variety of geometric shapes and sizes. Initially, a fenestrated mesh implant may not be as strong as a solid sheet implant. However, because of the increased surface area and the potential for fibrovascular infiltration through the fenestrations, a fenestrated implant will ultimately be stronger than a solid sheet implant. The fenestrations may also be used to more securely anchor the mesh implant to the host.

In some implementations, the implant may be impregnated with a variety of agents including, but not limited to, antibiotics (e.g., penicillin, vancomycin, sulfa, and Cipro), anesthetics (e.g., lidocaine, bupivacaine, and tetracaine), steroids (e.g., Celestone, Depo-Medrol, prednisolone, methylprednisolone, and prednisone), growth factors, self-proliferating proteins, stem cells, mesenchymal cells, mesenchymal native cells, bone morphogenetic proteins, nutrients, topical antibiotics (e.g., Neosporin and polymyxin), or recombinant adenoviral vectors, which are capable of expressing tissue specific transcription factors. These agents may prevent infection and facilitate the healing process.

In one embodiment, a biocompatible alloplastic mesh implant is made into sheets that are rolled into a tube. The tubular form helps contain biological grafts such as allografts, tendon grafts, ligament grafts, autogenous tendon grafts, autogenous ligament grafts, and xenografts. In addition, once fibrovascular infiltration occurs, graft strength and stability will be significantly increased. The tubular form may come in a variety of lengths and diameters to accommodate different tendon and ligament thicknesses and lengths. In one embodiment, a tubular mesh implant is fenestrated to allow sutures, anchors, or staples to anchor the implant into the post tissue or into the graft material. When a biocompatible alloplastic mesh implant in a cylindrical tube is used for augmentation to ligaments or tendons, the average diameter of the cylindrical tube will vary between 5 mm and 50 mm, the length will vary between 40 mm and 120 mm, and the thickness will be approximately 2 mm.

In another embodiment, a biocompatible alloplastic mesh implant serves as a biological bandage over conditions such as venostasis ulcers, diabetic ulcers, areas of skin breakdown, and soft tissue defects of the integumentary system. In one embodiment, a mesh bandage has a circular shape with a radius between 10 mm and 100 mm and a thickness between 1 mm and 10 mm, with an optimal 20 mm radius and 3 mm thickness. The mesh bandage may be fenestrated if the dressing needs to be anchored at its periphery or in the center with sutures, anchors or staples. In one embodiment, a mesh bandage is impregnated with a plurality of agents to facilitate the healing process including, but not limited to, antibiotics (e.g., penicillin, vancomycin, sulfa, and Cipro), anesthetics (e.g., lidocaine, bupivacaine, and tetracaine), steroids (e.g., Celestone, Depo-Medrol, prednisolone, methylprednisolone, and prednisone), growth factors, self-proliferating proteins, stem cells, mesenchymal cells, mesenchymal native cells, bone morphogenetic proteins, nutrients, topical antibiotics (e.g., Neosporin and polymyxin), or recombinant adenoviral vectors, which are capable of expressing specific transcription factors.

A mesh implant comprising a biocompatible alloplastic bulking agent provides many important advantages. Many mesh implants are sewn into attenuated fibers of the muscle, aponeurosis, or ligament and are only as strong as the tissue in which the mesh is sewn into. When the implant includes microparticles, the body's own biological response for fibrous tissue for healing occurs especially quickly along the scaffold of the implant. The particles make this process occur much faster than mesh implants without particles associated therewith. The body's inflammatory response to the mesh implant is such that fibrous tissue forms a capsule around the biological mesh, and, thus, provides stability and security in the repair. The risk of infection with biocompatible alloplastic mesh implants is significantly decreased, and irrigation, debridement, and antibiotics may be the only treatment needed to prevent infection.

Various modifications to these examples may be readily apparent to those skilled in the art, and the principles defined herein may be applied to other examples without departing from the spirit or scope of the novel aspects described herein. Thus, the scope of the disclosure is not intended to be limited to the examples shown herein but is to be accorded the widest scope consistent with the principles and novel features disclosed herein. Accordingly, the novel aspects described herein is to be defined solely by the scope of the following claims.

What is claimed is:

1. An implant comprising:
a sheet material; and
a plurality of microparticles in association with the sheet material.

2. The implant of claim 1, comprising collagen.

3. The implant of claim 1, comprising a fibrous sheet coated with a mixture of collagen and microparticles.

4. The implant of claim 1, wherein the implant is fenestrated.

5. The implant of claim 1, wherein the implant is not fenestrated.

6. The implant of claim 1, wherein the implant is tubular.

7. The implant according to claim 1, wherein the implant is a bandage.

8. The implant according to claim 1, wherein the implant is impregnated with at least one additional therapeutic agent.

9. The implant according to claim 6 wherein the agents are chosen from a group consisting of antibiotics, anesthetics, steroids, growth factors, self-proliferating proteins, mesenchymal cells, mesenchymal native cells, stem cells, topical antibiotics, bone morphogenetic proteins, and recombinant adenoviral vectors.

10. The implant of claim 1, wherein the particles comprise polymethylmethacrylate.

11. The implant of claim 1, wherein the ratio of particles to collagen is 1:1 to 1:5.

12. The implant of claim 1, wherein the collagen comprises bovine collagen.

13. The implant of claim 1, wherein the implant comprises 0.3% lidocaine.

14. The implant of claim 9, wherein the particles comprise microspheres.

15. A method of treating an ulcer comprising applying the implant of claim 1 to an ulcer.

16. A method of treating a skin defect comprising applying the implant of claim 1 to a skin defect.

17. A method of treating a hernia comprising applying the implant of claim 1 to a hernia.

18. A method of making an implant comprising coating a sheet material with a substance comprising a plurality of microparticles in a suspending agent.

19. The method of claim 18, wherein the suspending agent comprises collagen.
